(19) United States
(12) Patent Application Publication
(10) Pub. No.: US 2019/0093132 A1
(43) Pub. Date: Mar. 28, 2019

(54) METHOD OF MANUFACTURING CHEMICAL AND METHOD OF CULTURING MICROORGANISM

(71) Applicant: Toray Industries, Inc., Tokyo (JP)

(72) Inventors: Kenji Sawai, Tokyo (JP); Takashi Mimitsuka, Kamakura (JP); Kaoru Amagai, Kamakura (JP); Shota Sekiguchi, Kamakura (JP); Katsushige Yamada, Kamakura (JP)

(21) Appl. No.: 16/085,919

(22) PCT Filed: Mar. 16, 2017
(86) PCT No.: PCT/JP2017/010555

§ 371 Date: Sep. 17, 2018

(30) Foreign Application Priority Data
Mar. 17, 2016 (JP) 2016-053831

(51) Int. Cl. C12P 7/06

(52) U.S. Cl. CPC C12P 7/06

(57) ABSTRACT

A method of producing a chemical product includes culturing a microorganism with a fermentation feedstock containing cane molasses as a main component; filtering the resulting culture liquid through a separation membrane to recover a filtrate which contains the chemical product and from which the microorganism has been separated; retaining or returning an unfiltered liquid containing the microorganism, in or to the culture liquid; and adding an additional fermentation feedstock to the culture liquid to carry out continuous fermentation; in which the microorganism cultured causes a centrifugal supernatant of the culture liquid to contain particles having an average particle diameter of 100 nm or more.

### TECHNICAL FIELD

This disclosure relates to a method of producing a chemical product by continuous culture with a fermentation feedstock containing cane molasses as a main component.

### BACKGROUND

As the problem of carbon dioxide emission into the atmosphere and the energy problem have been actualized, biomass-derived chemical products represented by biodegradable polymer materials such as lactic acid and biofuels such as ethanol have attracted stronger attention as products with sustainability and life cycle assessment (LCA) capability. These biodegradable polymer materials and biofuels are generally produced as fermentation products from microorganisms in a method in which, as a fermentation feedstock, glucose, which is a hexose, purified from edible biomass such as maize is used, or cane molasses generated in the process of purifying sugar from sugar cane is used. Cane molasses is consumed in large quantities as an ethanol fermentation feedstock and serves as an important fermentation feedstock in sugar-producing countries such as Brazil, Thailand and the like.

Examples of common methods of producing chemical products by microorganism culture include batch culture, fed-batch culture, continuous culture and the like. WO 2007/097260 discloses that the production rate and yield of a chemical product which is a fermentation product are enhanced by continuous culture using a separation membrane. However, WO 2007/097260 includes no description of the use of a cane-molasses-containing feedstock. In addition, WO 2012/118171 discloses that adding cane molasses after enzymatic saccharification of pretreated biomass enhances the yield of the saccharifying enzyme recovered through the membrane, and discloses a method of producing ethanol by microorganism fermentation using the obtained sugar liquid as a feedstock.

We studied continuous culturing based on utilizing a separation membrane and using a fermentation feedstock containing cane molasses as a main component and, consequently, found a new problem in that the continuous culture causes membrane clogging even at a rate of filtration (flux) that does not cause membrane clogging with a fermentation feedstock containing no cane molasses. In view of this, it could be helpful to provide a method that enables the use of a fermentation feedstock containing cane molasses as a main component, by which the same separation-membrane-utilized continuous culture is achieved when using a fermentation feedstock containing no cane molasses.

### SUMMARY

We culture microorganisms that cause the centrifugal supernatant of a culture liquid to contain microorganism-derived particles having an average particle diameter of 100 nm or more in separation-membrane-utilized continuous fermentation with a fermentation feedstock containing cane molasses as a main component.

We thus provide (1) to (5):

(1) A method of producing a chemical product, including the steps of: culturing a microorganism with fermentation feedstock containing cane molasses as a main component; filtering the resulting culture liquid through a separation membrane to recover a filtrate which contains the chemical product and from which the microorganism has been separated; retaining or returning an unfiltered liquid containing the microorganism, in or to the culture liquid; and adding an additional fermentation feedstock to the culture liquid to carry out continuous fermentation; wherein the microorganism cultured causes a centrifugal supernatant of the culture liquid to contain particles having an average particle diameter of 100 nm or more.
(2) The method of producing a chemical product according to (1), wherein the particles have an average particle diameter of 300 nm or more.
(3) The method of producing a chemical product according to (1) or (2), wherein the fermentation feedstock includes a mixture of cane molasses and a sugar liquid derived from a cellulose-containing biomass.
(4) The method of producing a chemical product according to any one of (1) to (3), wherein the microorganism is a yeast belonging to the genus Schizosaccharomyces.
(5) A method of culturing a microorganism, including the steps of: culturing a microorganism with a fermentation feedstock containing cane molasses as a main component; filtering the resulting culture liquid through a separation membrane; retaining or returning an unfiltered liquid containing the microorganism, in or to the culture liquid; and adding an additional fermentation feedstock to the culture liquid to carry out continuous culture; wherein the microorganism cultured causes a centrifugal supernatant of the culture liquid to contain particles having an average particle diameter of 100 nm or more.

We enable prevention of membrane clogging of a separation membrane even in separation-membrane-utilized continuous fermentation using a cane-molasses-containing fermentation feedstock and enable efficient production of a chemical product.

### BRIEF DESCRIPTION OF THE DRAWINGS

FIG. 1 shows changes in the filtration flux and the transmembrane pressure difference in separation-membrane-utilized continuous fermentation of a cane-molasses-containing feedstock using the Schizosaccharomyces pombe NBRC1628 strain.

FIG. 2 shows changes in the filtration flux and the transmembrane pressure difference in separation-membrane-utilized continuous fermentation of a cane-molasses-containing feedstock using the Saccharomyces cerevisiae NBRC2260 strain.

FIG. 3 shows changes in the filtration flux and the transmembrane pressure difference in continuous filtration using a cane-molasses-containing feedstock.

FIG. 4 shows changes in the filtration flux and the transmembrane pressure difference in separation-membrane-utilized continuous fermentation of a cane-molasses-containing feedstock using the Schizosaccharomyces japonicus NBRC1609 strain.

FIG. 5 shows changes in the filtration flux and the transmembrane pressure difference in separation-membrane-utilized continuous fermentation of a feedstock containing no cane molasses using the Saccharomyces cerevisiae NBRC2260 strain.

### DETAILED DESCRIPTION

We provide a method of producing a chemical product and a method of culturing a microorganism which are characterized by including the steps of: culturing a microorganism with a fermentation feedstock containing cane molasses as a main component; filtering the resulting culture liquid through a separation membrane to recover a filtrate which contains the chemical product and from which the microorganism has been separated; retaining or returning an unfiltered liquid containing the microorganism, in or to the culture liquid; and adding an additional fermentation feedstock to the culture liquid to carry out continuous fermentation; in which the microorganism cultured causes a centrifugal supernatant of the culture liquid to contain particles having an average particle diameter of 100 nm or more.

A microorganism used in the methods has the capability to produce a chemical product and, without particular limitation, may be any microorganism that causes the centrifugal supernatant of a culture liquid to contain particles having an average particle diameter of 100 nm or more when the microorganism is cultured with a fermentation feedstock containing cane molasses as a main component. Specific preferable examples of such microorganisms include yeasts belonging to the genus Shizosaccharomyces. As a yeast belonging to the genus Shizosacharomyces, Shizosaccharomyces pombe, Shizosaccharomyces japonicus, Shizosaccharomyces octosporus, or Shizosaccharomyces cryophilus can be suitably used.

A “particle” refers to an insoluble particulate substance other than a microorganism contained in a culture liquid. The average particle diameter of particles present in a culture liquid is measured by dynamic light scattering (DLS, photon correlation method). Specifically, an autocorrelation function is determined by cumulant analysis from a fluctuation in the scattering intensity obtained by measurement using dynamic light scattering, and the autocorrelation function is converted to a particle size distribution relative to the scattering intensity and then converted to an average particle diameter in the analysis range from the minimum value of 1 nm to the maximum value of 5000 nm. For the measurement, the ELS-Z2 made by Otsuka Electronics Co., Ltd. is used. In addition, because the microorganism is present also as particles in the culture liquid, the culture liquid at room temperature is centrifuged under the conditions at 1000×G for 10 minutes to deposit the microorganism, and the average particle diameter of the particles contained in the centrifugal supernatant is measured.

The particles have an average particle diameter of 100 nm or more, preferably 300 nm or more, more preferably 300 to 1500 nm. Use of a microorganism that causes a culture liquid to contain such particles having an average particle diameter of 100 nm or more enables remarkable suppression of membrane clogging of a separation membrane as illustrated in the below-mentioned Examples and Comparative Examples, although the detailed action mechanism is not clear. In this regard, the upper limit of the average particle diameter of particles is not limited to a particular value to the extent that the filtration flux is not reduced by the occurrence of membrane clogging, but the upper limit is the average particle diameter of such particles as do not deposit together with a microorganism through the centrifugation, and the preferable upper limit value is 1500 nm.

Cane molasses is a byproduct produced in the process of sugar production from sugar cane squeezed juice or raw sugar. In other words, cane molasses refers to a crystallization mother liquor containing a sugar component remaining after crystallization in a crystallization step in a sugar production process. In general, the crystallization step is carried out usually a plurality of times, in which crystallization is repeated to go through the first crystallization carried out to afford a crystal component as the first sugar, further crystallization of the residual liquid (the first molasses) from the first sugar to afford a crystal component as the second sugar, still further crystallization of the residual liquid (the second molasses) from the second sugar to afford the third sugar and so on, and the molasses obtained at the final stage as a crystallization mother liquor remaining from the step is called cane molasses. As the number of times of crystallization increases, inorganic salts other than sugar components are more concentrated in cane molasses. As cane molasses, cane molasses that has undergone crystallization many times is preferable, and cane molasses remaining after crystallization is carried out at least two times or more, more preferably three times or more, is preferable. The sugar components contained in cane molasses include sucrose, glucose, and fructose as main components, and may include other sugar components in slight amounts such as xylose and galactose. The sugar concentration of cane molasses is generally about 200 to 800 g/L. The sugar concentration of cane molasses can be quantified by a known measurement technique such as HPLC.

A fermentation feedstock means that which contains all nutrients required to grow microorganisms. The fermentation feedstock only needs to contain cane molasses as a main component and, in addition, carbon sources, nitrogen sources, inorganic salts and, if necessary, organic micronutrients such as amino acids and vitamins may be suitably added. In this regard, a fermentation feedstock containing cane molasses as a main component means that 50 weight percent or more of the matter (not including water) contained in the fermentation feedstock is cane molasses.

Examples of carbon sources to be preferably used include; saccharides such as glucose, sucrose, fructose, galactose, and lactose; starch saccharified liquids containing these sugars; sweet potato molasses, sugar beet molasses, and high test molasses; furthermore, organic acids such as acetic acids; alcohols such as ethanol; glycerin; and besides, sugar liquids derived from cellulose-containing biomass.

Examples of cellulose-containing biomass include: trees/plants-based biomass such as bagasse, switchgrass, corn stover, rice straw, and wheat straw; wood-based biomass such as trees and waste construction materials; and the like. Cellulose-containing biomass contains cellulose or hemicellulose which is a polysaccharide resulting from dehydration condensation of sugar, and hydrolysis of such a polysaccharide allows production of a sugar liquid usable as a fermentation feedstock.

A method of preparing a sugar liquid derived from cellulose-containing biomass is not limited to a particular one, and examples of disclosed methods of producing such a sugar include: a method in which a sugar liquid is produced by acid hydrolysis of biomass using a concentrated sulfuric acid (JPH11-506934W, JP2005-229821A); and a method in which a sugar liquid is produced by hydrolysis treatment of biomass using a diluted sulfuric acid and then further by enzymatic treatment using cellulase or the like (A. Aden, “Lignocellulosic Biomass to Ethanol Process Design and Economics Utilizing Co-Current Dilute Acid Prehydrolysis and Enzymatic Hydrolysis for Corn Stover”, NREL Technical Report (2002)). In addition, examples of disclosed methods in which no acid is used include: a method in which a sugar liquid is produced by hydrolysis of biomass using subcritical water in the order of 250 to 500° C. (JP2003-212888A); a method in which a sugar liquid is produced by subcritical water treatment of biomass and then further by enzymatic treatment (JP2001-95597A); and a method in which a sugar liquid is produced by hydrolysis treatment of biomass in the order of 240 to 280° C. using hot water under pressure and then further by enzymatic treatment (JP3041380B). After the above-mentioned treatments, the obtained sugar liquid and cane molasses may be mixed and purified. Such a method is disclosed in, for example, WO2012/118171.

Examples of nitrogen sources to be used include: ammonia gas, ammonia water, ammonium salts, urea, and nitric acid salts; other organic nitrogen sources to be supplementarily used, for example, oil cakes, soya bean hydrolysate liquids, casein degradation products, and other amino acids, vitamins, corn steep liquors, yeasts or yeast extracts, meat extracts, peptides such as peptone, various fermentation microbial cells and hydrolysates thereof; and the like.

As an inorganic salt, phosphate, magnesium salt, calcium salt, iron salt, manganese salt or the like can be suitably added, if necessary.

In addition, when a microorganism requires a specific nutrient to grow, the nutritive substance can be added as a standard sample or a natural product containing the substance.

The separation membrane is not limited to a particular one and may be any of those which have the function of separating, from a microorganism by filtration, a culture liquid obtained by microorganism culture, and examples of usable materials include porous ceramic membranes, porous glass membranes, porous organic polymer membranes, metallic fiber textiles, nonwoven fabrics and the like, among which particularly porous organic polymer membranes or ceramic membranes are preferred.

In view of resistance to dirt, the separation membrane is preferably structured, for example, as a separation membrane containing a porous resin layer as a functional layer.

The separation membrane having a porous resin layer preferably has, on the surface of a porous base material, a porous resin layer that acts as a separation function layer. The porous base material supports the porous resin layer to give strength to the separation membrane. When the separation membrane has a porous resin layer on the surface of a porous base material, the porous base material may be impregnated with the porous resin layer, or may not be impregnated with the porous resin layer.

The average thickness of the porous base material is preferably 50 to 3000 μm.

The porous base material is composed of an organic material and/or inorganic material and/or the like, and an organic fiber is preferably used. Examples of preferred porous base materials include woven fabrics and nonwoven fabrics composed of organic fibers such as cellulose fibers, cellulose triacetate fibers, polyester fibers, polypropylene fibers and polyethylene fibers, and more preferably, nonwoven fabrics are used because their density can be relatively easily controlled, they can be simply produced, and they are inexpensive.

As the porous resin layer, an organic polymer membrane can be preferably used. Examples of organic polymer membrane materials include polyethylene resins, polypropylene resins, polyvinyl chloride resins, polyvinylidene fluoride resins, polysulfone resins, polyethersulfone resins, polyacrylonitrile resins, cellulose resins, cellulose triacetate resins and the like. The organic polymer membrane may be a resin mixture containing these resins as main components. The main component means that the component is contained in an amount of 50 wt % or more, preferably 60 wt % or more. Examples of preferred organic polymer membrane materials include those which can be easily formed into a membrane using a solution and have excellent physical durability and chemical resistance such as polyvinyl chloride resins, polyvinylidene fluoride resins, polysulfone resins, polyethersulfone resins and polyacrylonitrile resins, and polyvinylidene fluoride resins or resins containing them as a main component are most preferably used.

As the polyvinylidene fluoride resin, a homopolymer of vinylidene fluoride is preferably used. Further, as the polyvinylidene fluoride resin, a copolymer of vinylidene fluoride and a vinyl monomer capable of copolymerizing therewith is also preferably used. Examples of vinyl monomers capable of copolymerizing with vinylidene fluoride include tetrafluoroethylene, hexafluoropropylene, ethylene fluoride trichloride and the like.

The separation membrane has only to have a pore size that does not allow the passage of the microorganism used in the fermentation, and the pore size is desirably in a range such that the separation membrane is less likely to suffer clogging due to secretions of the microorganism used in the fermentation and fine particles in the fermentation feedstock, and stably maintains its filtration performance for a long time. Thus, the average pore size of the porous separation membrane is preferably 0.01 to 5 μm. When the average pore size of the separation membrane is 0.01 to 1 μm, both a high blocking rate which does not allow leakage of the microorganism and high water permeability can be achieved, and the water permeability can be maintained for a long time.

The average pore size of the separation membrane is preferably 1 μm or less because, when the average pore size is close to the size of the microorganism, the pores may be directly clogged with the microorganism. In view of preventing leakage of the microorganism, that is, preventing the occurrence of a trouble causing a decrease in the blocking rate, the average pore size of the separation membrane is preferably not too large relative to the size of the microorganism. When bacteria whose cells are small or the like are used as the microorganism, the average pore size is preferably 0.4 μm or less, more preferably 0.2 μm or less, still more preferably 0.1 μm or less. Too small an average pore size reduces the water permeability of the separation membrane, which then does not enable efficient operation even though the separation membrane is not fouled, and accordingly the average pore size of the separation membrane is preferably 0.01 μm or more, more preferably 0.02 μm or more, still more preferably 0.04 μm or more.

The average pore size can be determined by measuring the diameters of all pores observed within an area of 9.2 μm×10.4 μm under a scanning electron microscope at a magnification of 10,000×, and averaging the measured values. Alternatively, the average pore size can be determined by: taking a picture of the surface of a membrane using a scanning electron microscope at a magnification of 10,000×; randomly selecting 10 or more pores, preferably 20 or more pores; measuring the diameters of these pores; and calculating the number average. When the pore is not circular, a circle having the same area as the pore has (equivalent circle) can be determined using an image processing device or the like, and the diameter of the equivalent circle is regarded as the diameter of the pore.

The standard deviation σ of the average pore size of the separation membrane is preferably 0.1 μm or less. The smaller the standard deviation σ of the average pore size, the better. The standard deviation σ of the average pore size is calculated according to the following Equation (1), wherein n represents the number of pores observable within the above-mentioned area of 9.2 μm×10.4 μm; Xk represents the respective measured diameters; and X(ave) represents the average of the pore sizes.

$$σ = \sqrt{\frac{ {\sum }_{k = 1}^{N} {( {X}_{k} - X ( ave ) )}^{2}}{N}} \qquad ( 1 )$$

The permeability of the separation membrane for a fermentation culture liquid is one of its important properties. As an index of the permeability of the separation membrane, the pure water permeability coefficient of the separation membrane before use can be used. The pure water permeability coefficient of the separation membrane is preferably 5.6×10⁻¹⁰ m³/m²/s/pa or more, as calculated when the amount of water permeation is measured at a head height of 1 m using purified water having a temperature of 25° C. prepared by filtration through a reverse osmosis membrane. When the pure water permeability coefficient is from 5.6×10⁻¹⁰ m³/m²/s/pa to 6×10⁻⁷ m³/m²/s/pa, a practically sufficient amount of water permeation can be obtained.

The surface roughness of the separation membrane means the average height in the direction perpendicular to the surface. The membrane surface roughness is one of the factors which enable the microorganism adhering to the surface of the separation membrane to be detached more easily by the membrane surface washing effect of a liquid current generated by stirring or a circulating pump. The surface roughness of the separation membrane is not limited to a particular value, but has only to be in a range such that the microorganism and other solids adhering to the membrane can be detached, and the surface roughness is preferably 0.1 μm or less. When the surface roughness is 0.1 μm or less, the microorganism and other solids adhering to the membrane can be easily detached.

The separation membrane more preferably has a surface roughness of 0.1 μm or less, an average pore size of 0.01 to 1 μm, and a pure water permeability coefficient of 2×10⁻⁹ m³/m²/s/pa or more, and using such a separation membrane has revealed that the operation can be more easily carried out thereby without requiring excessive power for washing the membrane surface. When the separation membrane surface roughness is 0.1 μm or less, the shear force generated on the membrane surface can be reduced during the filtration of the microorganism, destruction of the microorganism can be suppressed, and clogging of the separation membrane can be suppressed so that stable filtration can be more easily carried out for a long time. When the membrane surface roughness of the separation membrane is 0.1 μm or less, continuous fermentation can be carried out with a smaller transmembrane pressure difference and, even when the separation membrane is clogged, the membrane can be more easily recovered by washing compared to when the operation is carried out with a large transmembrane pressure difference. Because suppressing the clogging of the separation membrane enables stable continuous fermentation, the surface roughness of the separation membrane is preferably as small as possible.

The membrane surface roughness of the separation membrane is measured using the following atomic force microscope (AFM) under the following conditions:
Apparatus: atomic force microscope apparatus (“Nanoscope Ma” made by Digital Instruments)
Conditions: Probe: SiN cantilever (made by Digital Instruments)
   Scanning mode: contact mode (measurement in air)
      Underwater tapping mode (underwater measurement)
   Scanning area: 10 μm square, 25 μm square (measurement in air)
      5 μm square, 10 μm square (underwater measurement)
   Scanning resolution: 512×512
Sample preparation: for the measurement, the membrane sample was soaked in ethanol at room temperature for 15 minutes, and then soaked in RO water for 24 hours, followed by washing and drying it in the air. The RO water means water prepared by filtration through a reverse osmosis membrane (RO membrane), which is a filtration membrane, to remove impurities such as ions and salts. The pore size of the RO membrane is about 2 nm or less.

The membrane surface roughness, drough, is calculated according to the following Equation (2) on the basis of the height of each point in the direction of the Z-axis using the above atomic force microscope apparatus (AFM).

$${d}_{rough} = {\sum }_{n = 1}^{N} \frac{\vert {Z}_{n} - \overline{Z} \vert}{N} \qquad ( 2 )$$

d_{rough}: Surface Roughness (μm)
Zₙ: Height in the direction of Z-axis (μm)
   : Average Height in Scanning Area (μm)
N: Number of Measurement Samples

The separation membrane is not limited to a particular shape, but a flat membrane, a hollow fiber membrane or the like can be used, and a hollow fiber membrane is preferable. When the separation membrane is a hollow fiber membrane, the inner diameter of the hollow fiber is preferably 200 to 5000 μm, and the membrane thickness is preferably 20 to 2000 μm. Textile or knit produced by forming an organic fiber or an inorganic fiber into a cylindrical shape may be contained in the hollow fiber.

The above-mentioned separation membrane can be produced by, for example, the production method described in WO2007/097260.

The continuous fermentation is characterized by including the steps of: filtering a culture liquid for a microorganism through a separation membrane to recover a filtrate which contains a chemical product and from which the microorganism has been separated; retaining or returning an unfiltered liquid containing the microorganism in or to the culture liquid; and adding an additional fermentation feedstock to the culture liquid to carry out the continuous fermentation, in which the product is recovered from the filtrate.

In the method of producing a chemical product, the transmembrane pressure difference during the filtration is not limited to a particular value, but is acceptable as long as the filtration of the fermentation culture liquid is possible. However, when filtration treatment using an organic polymer membrane with a transmembrane pressure difference of more than 150 kPa is carried out to filter a culture liquid, the structure of the organic polymer membrane is more likely to be destroyed, and this may lead to the lowered capability to produce the chemical product. In addition, with a transmembrane pressure difference of less than 0.1 kPa, the amount of water permeation of the fermentation culture liquid is often insufficient so that the productivity in production of the chemical product tends to be low. Thus, in the method of producing a chemical product, a transmembrane pressure difference preferably of 0.1 to 150 kPa as the filtration pressure is used for an organic polymer membrane, whereby the amount of permeation of the fermentation culture liquid is large, and there is no lowering of the capacity to produce the chemical product due to destruction of the membrane structure so that the capability to produce the chemical product can be kept high. In organic polymer membranes, the transmembrane pressure difference is preferably 0.1 to 50 kPa, more preferably 0.1 to 20 kPa.

The temperature during the fermentation by the yeast can be set to a temperature suitable for the yeast used, and is not limited to a particular value as long as it is within the range in which the microorganism can grow, and the fermentation is carried out at 20 to 75° C.

In the method of producing a chemical product, batch culture or fed-batch culture may be carried out in the initial phase of the culture to increase the microorganism concentration and, after this, continuous fermentation (filtration of the culture liquid) may be started. Alternatively, microbial cells at a high concentration may be seeded, and continuous fermentation may be started at the beginning of culture. In the method of producing a chemical product, it is possible to start supply of the fermentation feedstock and filtration of the culture liquid at appropriate timings. The times to start the supply of the fermentation feedstock and filtration of the culture liquid do not necessarily need to be the same. In addition, the supply of the culture medium and the filtration of the culture liquid may be carried out either continuously or intermittently.

Nutrients necessary for growth of the microbial cells may be added to the fermentation feedstock supply to allow continuous growth of the microbial cells. The microorganism concentration of the culture liquid is a concentration preferred to achieve efficient productivity so that the productivity of the chemical product can be maintained at a high level. A good production efficiency can be obtained by maintaining the microorganism concentration of the culture liquid at, for example, 5 g/L or more in terms of dry weight.

In the method of producing a chemical product, a part of the culture liquid containing the microorganism may be removed from the fermenter, if necessary, during the continuous fermentation, and the culture liquid may then be supplied with fermentation feedstock and thus diluted to thereby control the concentration of the microorganism in the culture vessel. For example, if the concentration of the microbial cells in the fermenter is too high, clogging of the separation membrane is likely to occur and, in view of this, clogging may be prevented by removing a part of the culture liquid containing the microorganism and diluting the culture liquid with a fermentation feedstock supplied. In the method of producing a chemical product, the number of fermenters does not matter.

The continuous fermentation device is not limited to a particular one as long as it is a chemical product production device based on continuous fermentation including the steps of: filtering a culture liquid for a yeast through a separation membrane to recover a product from a filtrate; retaining or returning an unfiltered liquid containing the microorganism, in or to the culture liquid; and adding an additional fermentation feedstock to the culture liquid, in which the product is recovered from the filtrate, and specific examples of usable devices include the devices described in WO2007/097260 and WO2010/038613.

Examples of chemical products produced by our methods include substances mass-produced in the fermentation industry such as alcohols, organic acids and the like. Examples of alcohols include ethanol, 1,3-propanediol, 1,3-butanediol, 2,3-butanediol, 1,4-butanediol, glycerol, butanol, isobutanol, 2-butanol, and isopropanol, and examples of organic acids include acetic acid, lactic acid, adipic acid, pyruvic acid, succinic acid, malic acid, itaconic acid, and citric acid. Further, our methods can also be applied to production of substances such as enzymes, antibiotics, and recombinant proteins. Such chemical products can be recovered from a filtrate by well-known methods (membrane separation, concentration, distillation, crystallization, extraction and the like).

In addition, our methods are not limited to the above-mentioned methods of producing a chemical product, but may be a culture method intended for growth of microorganisms in accordance with the above-mentioned methods. Specific examples of such methods include a culture method in which a microorganism is a product of interest.

### EXAMPLES

Below, our methods will specifically be described with reference to Examples. However, this disclosure is not to be limited thereto.

### Reference Example 1 Method of Analyzing Saccharides and Ethanol

The concentrations of saccharides and ethanol in the feedstock were quantified under the HPLC conditions described below, based on comparison with standard samples.

Column: Shodex SH1011 (made by Showa Denko K. K.)
Mobile phase: 5 mM sulfuric acid (flow rate: 0.6 mL/minute)
Reaction solution: none
Detection method: RI (differential refractive index)

### Temperature: 65° C.

### Reference Example 2 Preparation of Cane-Molasses-Containing Feedstock

A solid content obtained from hydrothermally-processed bagasse (C6 fraction) and water were mixed to make a liquid mixture with the solid content well-mixed at a concentration of 10%, and to this liquid mixture, 20 mg/g saccharifying enzyme and dried bagasse were added so that the resulting mixture was allowed to react for saccharification for 48 hours. The saccharification reaction was carried out at 50° C. without pH control. To the resulting mixture, cane molasses was added so that the ratios shown in Table 1 could finally be reached in 48 hours and, subsequently, the resulting mixture was subjected to solid-liquid separation between a saccharification residue and a saccharified liquid, using a filter press, and then was allowed to pass through the microfiltration membrane and the ultrafiltration membrane to obtain a cane-molasses-containing feedstock. The cane-molasses-containing feedstock analysis results obtained using the method shown in Reference Example 1 are shown in Table 2.

TABLE 1

| Fermentation Feedstock | Mixed Amount (wt %) |
|---|---|
| Hydrothermally-Processed | 7.7 |
| C6 Fraction | |
| Cane Molasses | 23.1 |

TABLE 2

| | Glucose | Fructose | Sucrose |
|---|---|---|---|
| | (g/L) | (g/L) | (g/L) |
| Cane-Molasses-Containing | 77.2 | 19.5 | 66.3 |
| Feedstock | | | |

### Example 1 Separation-Membrane-Utilized Continuous Fermentation Using Schizosaccharomyces pombe NBRC1628 Strain

Separation-membrane-utilized continuous culture was carried out using an ethanol producing yeast, the Schizosaccharomyces pombe NBRC1628 strain, as a culture microorganism and using, as a culture medium, the cane-molasses-containing feedstock shown in Table 2. A separation membrane element in the form of a hollow fiber described in JP2010-22321A was adopted. The Schizosaccharomyces pombe NBRC1628 strain was inoculated in a test tube in which 5 ml of the feedstock shown in Table 2 had been loaded and was subjected to shaking culture overnight (pre-pre-preculture). The obtained culture liquid was inoculated in an Erlenmeyer flask in which 45 ml of fresh feedstock shown in Table 2 had been loaded, and subjected to shaking culture at 30° C. at 120 rpm for eight hours (pre-preculture). Out of 50 mL of the pre-preculture liquid, 35 mL was taken, inoculated in a continuous fermentation device in which 700 mL of the cane-molasses-containing feedstock shown in Table 2 had been loaded, and stirred at 300 rpm using an accessory stirrer in a fermentation reaction vessel to be cultured for 24 hours (preculture). In this regard, a fermentation liquid circulating pump was started up immediately after the inoculation to cause liquid circulation between the separation membrane module and the fermenter. Upon completion of the preculture, a filtration pump was started up to start pulling the fermentation liquid out of the separation membrane module. After filtration was started, the fermentation feedstock was added so that the fermentation liquid in the continuous fermentation device could be controlled in an amount of 700 ml while continuous culture was carried out under the following continuous fermentation conditions for about 300 hours. The changes in the transmembrane pressure difference and the filtration rate in the continuous culture are shown in FIG. 1.

### Continuous Fermentation Conditions

Fermentation reaction vessel capacity: 2 (L)
Separation membrane used: filtration membrane made from polyvinylidene fluoride
Membrane separation element effective filter area: 218 (cm²)
Temperature adjustment: 30 (° C.)
Aeration rate in the fermentation reaction vessel: no aeration
Stirring rate in the fermentation reaction vessel: 300 (rpm)
pH adjustment: no adjustment
Filtration flux setting value: 0.1 (m³/m²/day)
Sterilization: the culture vessel including a separation membrane element was autoclaved at 121° C. for 20 minutes.
Average pore size: 0.1 μm
Standard deviation of average pore size: 0.035 μm
Membrane surface roughness: 0.06 μm
Pure water permeability coefficient: 50×10⁻⁹ m³/m²/s/pa

As shown in FIG. 1, the results show that, in the about-300-hour continuous culture, the transmembrane pressure difference was substantially constant, membrane clogging did not occur, and the filtration flux remained stable at a constant value. In addition, the ethanol concentration was 64 g/L at the point of time when the continuous culture was terminated. Comparative Example 1 Separation-Membrane-Utilized Continuous Fermentation 1 Using Saccharomyces cerevisiae NBRC2260 Strain

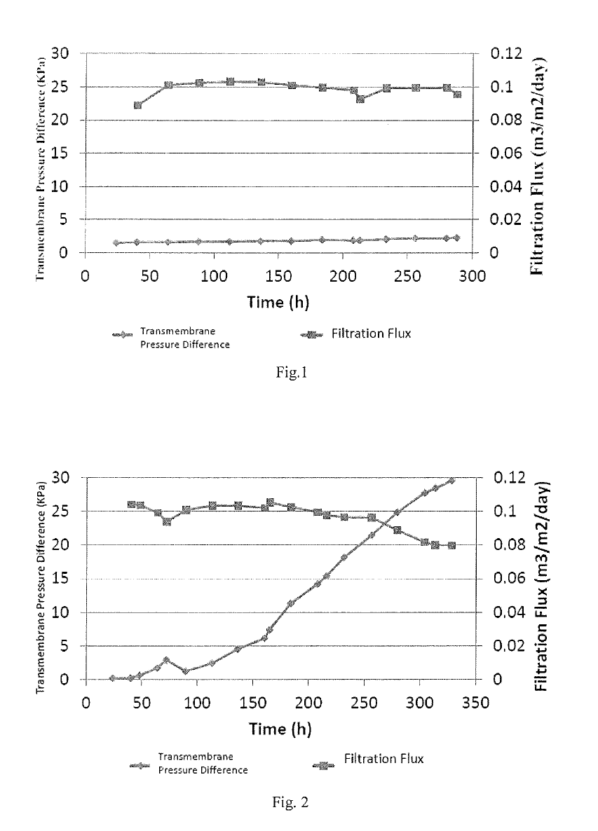

Continuous culture was carried out in the same manner as in Example 1 except that the Saccharomyces cerevisiae NBRC2260 strain, which is an ethanol-producing yeast, was used as a culture microorganism. The changes in the transmembrane pressure difference and the filtration flux in the continuous culture are shown in FIG. 2. FIG. 2 shows that, in the 300-hour continuous culture, the transmembrane pressure difference sharply rose after 100 hours elapsed, membrane clogging occurred, and thus the filtration flux went down below the setting value. In addition, the ethanol concentration was 65 g/L at the point of time when the continuous culture was terminated.

### Reference Example 3 Continuous Filtration Test Using Cane-Molasses-Containing Feedstock

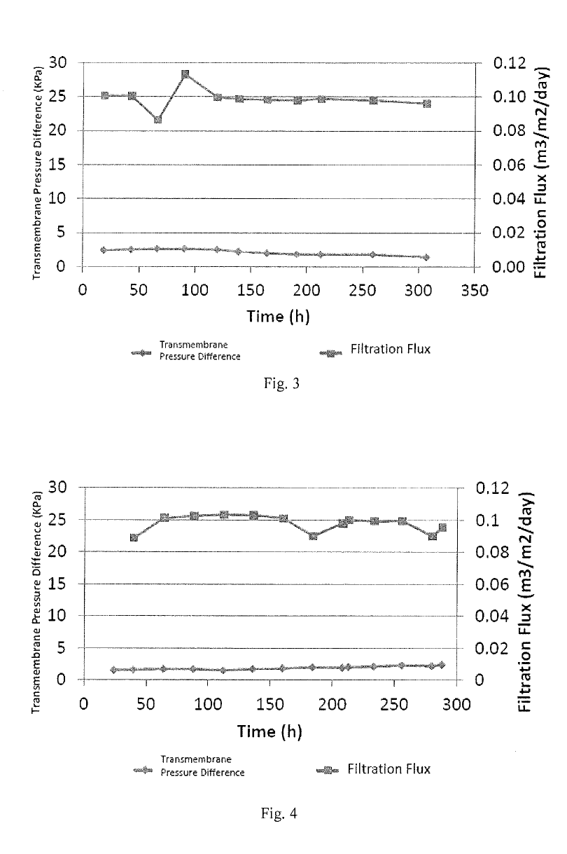

Next, a continuous filtration test in which the cane-molasses-containing feedstock shown in Table 2 was used singly was carried out. As to the temperature, rate of stirring, and pH for filtration, the test was carried out under the same conditions as in the method described in Example 1. FIG. 3 shows the changes in the transmembrane pressure difference and filtration flux in the continuous filtration test carried out for 600 hours. FIG. 3 shows that the transmembrane pressure difference was substantially constant, membrane clogging did not occur, and the filtration flux remained stable at a constant value.

### Example 2 Separation-Membrane-Utilized Continuous Fermentation Using Schizosaccharomyces japonicus NBRC1609 Strain

Continuous culture was carried out in the same manner as in Example 1 except that the Schizosaccharomyces japonicus NBRC1609 strain was used as a culture microorganism. The changes in the transmembrane pressure difference and the filtration flux in the continuous culture are shown in FIG. 4. FIG. 4 shows that, in the about-300-hour continuous culture, the transmembrane pressure difference was substantially constant, membrane clogging did not occur, and the filtration flux remained stable at a constant value.

### Reference Example 4 Separation-Membrane-Utilized Continuous Fermentation Test 2 Using Saccharomyces cerevisiae NBRC2260 Strain

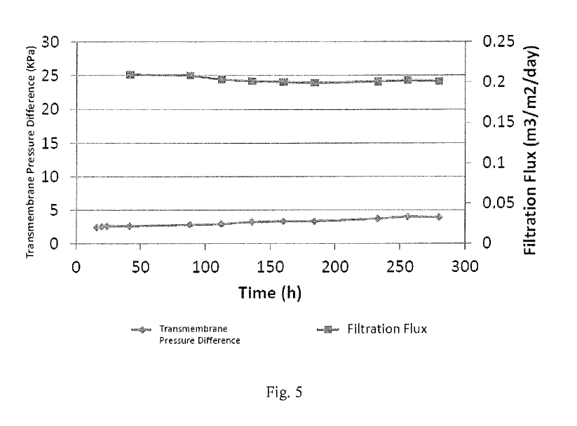

Continuous culture was carried out in the same manner as in Example 1 except that the Saccharomyces cerevisiae NBRC2260 strain, which is an ethanol-producing yeast, was used as a culture microorganism, and the feedstock containing no cane molasses shown in Table 3 was used as a fermentation feedstock. However, the test was carried out with the filtration flux set to 0.2 (m³/m²/day). The changes in the transmembrane pressure difference and the filtration rate in the continuous culture are shown in FIG. 5.

Although the setting filtration flux was twice as large as in Comparative Example 2, the results show that, in the about-300-hour continuous culture, the transmembrane pressure difference was substantially constant, membrane clogging did not occur, and the filtration flux remained stable at a constant value. In addition, the ethanol concentration was 47 g/L at the point of time when the continuous culture was terminated.

The results revealed that membrane clogging occurred or did not occur, depending on the combination of the fermentation feedstock materials or yeasts used.

TABLE 3

| Feedstock | Concentration (g/L) |
|---|---|
| Glucose | 100 |
| Yeast Nitrogen Base w/o amino acid and | 1.7 |
| ammonium sulfate (made by Difco) | |
| Ammonium Sulfate | 0.5 |

### Example 3 Measurement Results of Average Particle Diameter in Culture Liquid Supernatant

Each culture liquid and each cane-molasses-containing feedstock of Example 1, Example 2, Comparative Example 1, and Reference Example 3 were centrifuged, and the average particle diameter of the obtained supernatant was measured. Specifically, the Schizosaccharomyces pombe NBRC1628 strain or NBRC1609 strain or the Saccharomyces cerevisiae NBRC2260 strain was inoculated in a test tube to which 5 mL of the cane-molasses-containing feedstock of Reference Example 2 had been added, and cultured at 30° C. at 120 rpm for 72 hours. Each yeast culture liquid and the cane-molasses-containing feedstock of Reference Example 3 were centrifuged at 1000×G for 10 minutes, and the supernatants thereof each recovered in an amount of 3 mL. A 30 μL amount of the recovered supernatant was added to 970 μL of a pH5 citric acid buffer and thus diluted, and each diluted solution poured into a disposable cuvette having a capacity of 1 mL and measured by dynamic light scattering for average particle diameter.

### Measurement Conditions

Light source pinhole size: 100 μm
Measurement wavelength: 660 nm
Measurement angle: 165°
Measurement cumulated number: 70 times
Solvent refractive index: 1.3313
Solvent viscosity: 0.8852 cp

Next, the measurement results were analyzed under the following conditions.

### Analysis Conditions

For particle diameter analysis, a zeta-potential & particle size analyzer, ELS-Z2, made by Otsuka Electronics Co., Ltd. was used, and measurement was carried out in the air under 25° C. conditions. Specifically, an autocorrelation function was determined by cumulant analysis from a fluctuation in the scattering intensity obtained by dynamic light scattering, and the result converted to a particle size distribution relative to the scattering intensity. The histogram analysis range of the particle size distribution was from the minimum value of 1 nm to the maximum value of 5000 nm. The obtained average particle diameters are shown in Table 4.

TABLE 4

| | | | | Average | Standard | Membrane |
|---|---|---|---|---|---|---|
| | | | | Particle Size | Deviation | Clogging |
| | Microorganism | Fermentation Feedstock | Culture Method | [nm] | [nm] | present or not |
| Example 1 | Schizosaccharomyces | Cane-Molasses- | test tube culture | 366.4 | ±188.1 | not present |
| | pombe | Containing Feedstock | continuous | 1209 | ±579 | |
| | NBRC1628 strain | | fermentation | | | |
| Example 2 | Schizosaccharomyces | Cane-Molasses- | test tube culture | 316.4 | ±137.1 | not present |
| | japonicas | Containing Feedstock | continuous | 568 | ±248 | |
| | NBRC1609 strain | | fermentation | | | |
| Comparative | Saccharomyces cerevisiae | Cane-Molasses- | test tube culture | 13.9 | ±1.4 | present |
| Example 1 | NBRC2260 strain | Containing Feedstock | continuous | no particle | — | |
| | | | fermentation | | | |
| Reference | none | Cane-Molasses- | — | no particle | — | not present |
| Example 3 | | Containing Feedstock | | | | |

The results in Table 4 show that particles having an average particle diameter of 300 nm or more were included in the supernatant of the culture liquid of the Schizosaccharomyces pombe NBRC1628 strain and Schizosaccharomyces japonicus NBRC1609 strain with which no membrane clogging occurred and the filtration rate did not decrease in the continuous culture in which the separation membrane was used with the cane-molasses-containing feedstock. On the other hand, no particles having an average particle diameter of 300 nm or more were included in the supernatant of the culture liquid of the Saccharomyces cerevisiae NBRC2260 strain with which membrane clogging occurred and the filtration rate decreased in the continuous culture. In addition, no particles were found to be present in the cane-molasses-containing feedstock, either. That is, the results revealed that no membrane clogging occurs in the separation-membrane-utilized continuous fermentation in which a cane-molasses-containing culture medium is used and in which a microorganism that causes the centrifugal supernatant of a culture liquid to contain particles having an average particle diameter of 100 nm or more is used for fermentation.

1.-5. (canceled)

6. A method of producing a chemical product comprising:
culturing a microorganism with a fermentation feedstock containing cane molasses as a main component;
filtering the resulting culture liquid through a separation membrane to recover a filtrate containing said chemical product and from which said microorganism has been separated;
retaining or returning an unfiltered liquid containing said microorganism, in or to said culture liquid; and
adding an additional fermentation feedstock to said culture liquid to carry out continuous fermentation;
wherein said microorganism cultured causes a centrifugal supernatant of the culture liquid to contain particles having an average particle diameter of 100 nm or more.

7. The method according to claim 6, wherein said particles have an average particle diameter of 300 nm or more.

8. The method according to claim 6, wherein said fermentation feedstock comprises a mixture of cane molasses and a sugar liquid derived from a cellulose-containing biomass.

9. The method according to claim 6, wherein said microorganism is a yeast belonging to the genus Schizosaccharomyces.

10. A method of culturing a microorganism comprising:
culturing said microorganism with a fermentation feedstock containing cane molasses as a main component;
filtering the resulting culture liquid through a separation membrane;
retaining or returning an unfiltered liquid containing said microorganism, in or to said culture liquid; and
adding an additional fermentation feedstock to said culture liquid to carry out continuous culture;
wherein said microorganism cultured causes a centrifugal supernatant of said culture liquid to contain particles having an average particle diameter of 100 nm or more.

11. The method according to claim 7, wherein said fermentation feedstock comprises a mixture of cane molasses and a sugar liquid derived from a cellulose-containing biomass.

12. The method according to claim 7, wherein said microorganism is a yeast belonging to the genus Schizosaccharomyces.

13. The method according to claim 8, wherein said microorganism is a yeast belonging to the genus Schizosaccharomyces.
